(19) United States
(12) Patent Application Publication
(10) Pub. No.: US 2005/0148968 A1
(43) Pub. Date: Jul. 7, 2005

(54) Disposable absorbent garment including a stretchable carrier layer

(76) Inventors: Daniel R. Schlinz, Greenville, WI (US); Robert E. Vogt, Neenah, WI (US)

Correspondence Address: KIMBERLY-CLARK WORLDWIDE, INC., 401 NORTH LAKE STREET, NEENAH, WI, 54956, US

(21) Appl. No.: 10/750,188

(22) Filed: Dec. 30, 2003

(51) Int. Cl. A61F 13/15; A61F 13/20

(52) U.S. Cl. 604/378

(57) ABSTRACT

The present invention relates to disposable absorbent garments including a liquid barrier layer, a stretchable carrier layer, an elastic layer attached to the stretchable carrier layer and an absorbent section. The disposable absorbent garments include a front waist region, a back waist region and a crotch region that connects the front waist region and the back waist region. The front waist region, the back waist region and the crotch region each have a width in a lateral direction of the garments. The stretchable carrier layer has a width in the waist regions of about the width of the front waist region and the back waist region and a width in the crotch region of about the width of the crotch region.

### BACKGROUND OF THE INVENTION

Disposable absorbent garments, such as disposable diapers for infants and toddlers, training pants and adult incontinence garments are well known and their manufacture and sale have developed into well-established commercial markets. When they were first introduced, disposable absorbent garments were relatively simple and non-complex, with a minimum number of components. For example, early disposable diapers included an absorbent pad, surrounded on the clothing-facing side by a plastic film liquid barrier material and on the body-facing side by a tissue or nonwoven material. These early disposable diapers would also have included some mechanism for fastening one end of the product to the other when the product was applied to a child. Over time, with greater and greater consumer interest in disposable diapers over cloth diapers, additional features have been incorporated into disposable diaper designs. For example, leg and waist elastics, containment flaps and more complex fastening features have been adopted.

Generally speaking, disposable absorbent garments are sized to fit persons within a given weight range. For example, disposable diapers are sold in approximately seven different sizes to accommodate developmental weight ranges from newborn babies through toddlers that may be 3-4 years old. Not surprisingly, even within a given weight range, human bodies come in a wide variety of shapes and sizes. As a result, the design of disposable absorbent garments has gone in a direction of attempting to provide a greater range of “fit” by a single garment size, a challenging task. Better-fitting garments are not only better looking and more comfortable to wear, but they also tend to leak less because the various gasket features are able to perform their intended function.

The increased range of fit may be approached by introducing a variety of features including stretchable materials to form the chassis components (the “chassis” of a disposable absorbent garment is typically understood to include the outer cover or backsheet, the absorbent core or absorbent layer and the bodyside liner or topsheet), stretchable back “ear” portions to improve the range of engagement of the fasteners and stretchable waistband features. Currently-available disposable diapers, such as PAMPERS Custom Fit diapers manufactured and sold by the Procter & Gamble Co. of Cincinnati, Ohio and HUGGIES Supreme diapers manufactured and sold by the Kimberly-Clark Corporation of Neenah, Wis., include separately attached, stretchable back ear portions. Disposable absorbent garments generally include a front waist region, a back waist region and a crotch region that interconnects the front waist region and the back waist region. Disposable absorbent garments also generally define two longitudinal side edges and two waist edges. The stretchable back ear portions are attached to the longitudinal side edges of the chassis in the back waist region of the garments. The stretchable back ear portions are formed from elastically-stretchable nonwoven materials that are not used to form the chassis. Similarly, presently-available children's training pants are formed with elastomeric side panels to provide coverage over the hips of the wearer and to connect the front and back waist regions. As with disposable diapers, the elastomeric side panels are formed of elastic nonwoven materials that are separately attached to the chassis of the training pants. The stretchable back ear portions of diapers and elastomeric side panels of training pants may be attached to the garment chassis through the use of bonding techniques such as adhesive, ultrasonic or thermal bonding.

As may be generally appreciated, the incorporation of individual components onto the chassis increases the complexity of the manufacture of disposable absorbent garments. Every component that is attached to the chassis needs to be securely attached and needs to be provided at the correct location on the chassis (i.e. the component needs to be “registered” with the surrounding chassis components) in order to provide a functional and aesthetically-pleasing garment. Disposable absorbent garments are generally manufactured on relatively high speed production lines because their practicality drives high volumes of consumption. The more separate components that are introduced to the chassis during manufacture, the more likely that production rates will decrease.

Therefore, while it is desirable to increase the range of fit of disposable absorbent garments, there remains a need for product structures and processes that increase the simplicity with which stretchable components are incorporated. More specifically, there remains a need for a disposable absorbent garment that has a simplified construction and that eliminates the attachment of multiple separate components that increase the cost and complicate the manufacture of disposable absorbent garments. Additionally, there remains a need for flexible product structures and processes that increase the options for locations of stretchable materials within disposable absorbent garments.

### SUMMARY OF THE INVENTION

The present invention relates to a disposable absorbent garment and a method for making disposable absorbent garments. The disposable absorbent garments of the invention have a longitudinal direction and a lateral direction associated with them. The longitudinal direction is generally aligned with the length of the garment and the lateral direction is generally aligned with the width of the garment. The lateral direction is generally perpendicular to the longitudinal direction. The disposable absorbent garments may also include a front waist region, a back waist region and a crotch region that connects the front waist region and the back waist region. The waist regions are defined in terms of the areas that they cover when the garment is worn by a person. The crotch region connects the front waist region and the back waist region and generally falls between the legs of a wearer. The disposable absorbent garments may also include a waist region width in the lateral direction and a crotch region width in the lateral direction. The crotch region width may be less than the waist region width. As a result, the disposable absorbent garments may be wider in the waist regions than in the crotch regions. The disposable absorbent garments may also include two longitudinal side edges and two waist edges. The waist edges are generally oriented to be parallel with the lateral direction. The disposable absorbent garments may further include a liquid barrier layer, a stretchable carrier layer, an elastic layer and an absorbent section. The liquid barrier layer may include an inner surface and an outer surface. The liquid barrier layer may be generally planar and defines two generally planar surfaces (namely, the inner surface and the outer surface). The inner surface may generally correspond to the skin or body-facing surface of the garment and the outer surface may generally correspond to the clothing-facing surface of the garment. The liquid barrier layer may also define opposite longitudinal side edges and may have a liquid barrier layer width that lies between the opposite longitudinal side edges. The liquid barrier layer width may be approximately equal to the crotch region width.

The stretchable carrier layer may be placed in facing relationship with the liquid barrier layer. The stretchable carrier layer may have a width in the waist regions approximately equal to the waist region widths of the garment. Further, the stretchable carrier layer may have a width in the crotch region of the garment approximately equal to the crotch region width. The disposable absorbent garment may also include an elastic layer that is attached to the stretchable carrier layer. The elastic layer may have an elastic layer width. The elastic layer width may be defined as the distance extending away from the lateral centerline of the garment from a longitudinal side edge of the barrier layer to the nearest longitudinal side edge of the garment. The lateral centerline is an imaginary centerline that runs along the length of the garment to divide the width of the garment in half. Because the width of the elastic layer runs from a longitudinal side edge of the barrier layer to a longitudinal side edge of the disposable absorbent garment, the elastic layer does not generally overlap with the liquid barrier layer. Further, the elastic layer width runs from a longitudinal side edge of the barrier layer toward the longitudinal side edge of the garment that is one the same side of the lateral centerline (i.e. the nearest longitudinal side edge of the garment). The absorbent section of the disposable absorbent garment may be disposed on the inner surface of the liquid barrier layer. By being disposed on an inner surface of the liquid barrier layer, the absorbent section may be formed or applied directly or indirectly to the inner surface of the liquid barrier layer. Additionally, the absorbent section may be formed or applied between layers that are near the inner surface of the liquid barrier layer. The absorbent section may be formed or applied between layers that form the liquid barrier, formed or applied to a substrate that is placed with or near the liquid barrier layer, formed or applied within a layer of the liquid barrier layer or another substrate, or other variations or combinations thereof.

In another aspect, the present invention relates to a disposable absorbent garment for which the stretchable carrier layer defines an inner surface and an outer surface and the absorbent section is disposed on the inner surface of the stretchable carrier layer. Therefore, the layers of the garments working from the outside inward may include the liquid barrier layer, the stretchable carrier layer and then the absorbent section. In a separate aspect, the present invention relates to a disposable absorbent garment where the stretchable carrier layer includes an inner surface and an outer surface and the absorbent section is located between the inner surface of the liquid barrier layer and the outer surface of the stretchable carrier layer. With this aspect, the sequence of layers of the garment working from the outside inward are the liquid barrier layer, the absorbent section and the stretchable carrier layer. The stretchable carrier layer is then the layer that is in closest proximity to the skin of the wearer of the garment. With this construction, the elastic layer may be attached to the inner surface of the stretchable carrier layer. Alternatively, the elastic layer may be attached to the outer surface of the stretchable carrier layer. In another aspect, the present invention relates to a disposable absorbent garment that includes a stretchable containment flap layer that may be attached to the inner surface of the stretchable carrier layer. With this aspect, the elastic layer may be sandwiched between the inner surface of the stretchable carrier layer and the stretchable containment flap layer.

The disposable absorbent garments of the invention may have the elastic layer attached to the carrier layer on both sides of the lateral centerline in the back waist region of the garments. Therefore, an elastic layer may be attached running from the longitudinal side edge of the liquid barrier layer toward the longitudinal side edge of the garment on both sides of the liquid barrier layer. The resulting garment includes two elastic layers in the back waist region. Further, the disposable absorbent garment may include an elastic layer attached to the carrier layer on both sides of the lateral centerline in the back waist region and in the front waist region. The stretchable carrier layer may be extensible in the lateral direction of the garment. The elastic layer may have a fastener element attached to it so as to provide a fastening system for the garment. The fastener system may be used to secure the back waist region to the front waist region when the garment is worn.

In an additional aspect of the invention, there may be an elastic ear material bonded to a longitudinal side edge of the elastic layer. In this regard, the disposable absorbent garment would include an elastic layer followed by an elastic ear material going outward from a lateral centerline of the garment. The elastic ear material may have a fastener element attached. When an elastic ear material is bonded to the longitudinal side edge of the elastic layer, a fastening system may be provided that includes two sections of stretchable material to provide improved coverage between the waist regions of the garment.

In a further aspect, the present invention relates to a method of making a disposable absorbent garment that includes a front waist region, a back waist region and a crotch region that extends between and connects the waist regions. The disposable absorbent garment has a longitudinal direction aligned with the length of the garment and a lateral direction aligned with the width of the garment. The method includes a first step of providing a first continuous web where the first continuous web may include a stretchable carrier layer and intermittent elastic material segments. The elastic material segments may be provided in intermittent sections so that the stretchable carrier layer does not include continuous elastic segments. Second, a second continuous web may be provided where the second continuous web includes a liquid barrier material. The first continuous web may be joined in facing relationship with the second continuous web to form an outer cover web. The next step of the method of the invention may be to provide a series of absorbent assemblies that then may be attached to the outer cover web to form a garment web. The garment web may then be selectively cut into individual garments in such a way that the intermittent elastic material segments are generally bisected in a lateral direction of the garment. The method may further include a step of providing a liner web in facing relationship with the absorbent assemblies prior to the selective cutting of the garment web.

In an additional aspect the present invention relates to a method of making a disposable absorbent garment that includes a front waist region, a back waist region and a crotch region that extends between and connects the waist regions. The disposable absorbent garment also defines a longitudinal direction and lateral direction. The method includes a step of providing a first continuous web where the first continuous web may include a liquid barrier material and have an outer surface and an inner surface. Being generally planar, the liquid barrier material generally defines two flat surfaces. The method may further include a step of providing a series of absorbent assemblies and attaching the absorbent assemblies to the inner surface of the first continuous web. A second continuous web may be provided in the facing relationship with the inner surface of the first continuous web to form a garment web. The second continuous web may include a stretchable carrier layer and intermittent elastic material segments. The garment web may be selectively cut into individual garments in such a way that the intermittent elastic material segments are generally bisected in the lateral direction. The stretchable carrier layer may have an outer surface and an inner surface. The intermittent elastic material segments may be attached to the inner surface of the stretchable carrier layer. Alternatively, the stretchable carrier layer may have an outer surface and an inner surface and the intermittent elastic material segments may be attached to the outer surface of the stretchable carrier layer. The method may further include a step of providing a stretchable containment flap web in facing relationship with an inner surface of the stretchable carrier layer prior to the garment web being selectively cut.

These aspects and additional aspects of the invention will be described in greater detail herein. Further, it is to be understood that both the foregoing general description and the following detailed description are exemplary and are intended to provide further explanation of the invention claimed. The accompanying drawings, that are incorporated in and constitute part of this specification, are included to illustrate and provide a further understanding of the disposable absorbent articles of the invention. Together with the description, the drawings serve to explain various aspects of the invention.

### BRIEF DESCRIPTION OF THE DRAWINGS

The present invention will be more fully understood and further advantages will become apparent when reference is made to the following detailed description of the invention and the accompanying drawings wherein like numerals represent like elements. The drawings are merely representative and are not intended to limit the scope of the appended claims.

FIG. 1 representatively shows a plan view of an example of a disposable absorbent garment of the present invention (a garment to be worn by an infant/toddler) in an unfastened, stretched and laid flat condition with the surface of the garment that contacts the wearer's skin facing the viewer;

FIG. 2 representatively shows a cross-section view of the disposable absorbent garment of FIG. 1 through one of the waist regions of the garment;

FIG. 3 representatively shows another aspect of a cross-section view of the disposable absorbent garment of FIG. 1 through one of the waist regions of the garment;

FIG. 4 representatively shows another aspect of a cross-section view of the disposable absorbent garment of FIG. 1 through one of the waist regions of the garment;

FIG. 5 representatively shows another aspect of a cross-section view of the disposable absorbent garment of FIG. 1 through one of the waist regions of the garment; and

FIG. 6 representatively shows a plan view of the back waist region of another example of a disposable absorbent garment of the invention.

### DETAILED DESCRIPTION OF THE INVENTION

The present disclosure of the invention will be expressed in terms of its various components, elements, constructions, configurations, arrangements and other features that may also be individually or collectively be referenced by the term, “aspect(s)” of the invention, or other similar terms. It is contemplated that the various forms of the disclosed invention may incorporate one or more of its various features and aspects, and that such features and aspects may be employed in any desired, operative combination thereof.

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

The present invention is directed to solving problems related to providing structures for disposable absorbent garments and processes for making disposable absorbent garments having simplified incorporation of stretchable components. More specifically, the present invention is directed to eliminating the need for attachment of separate, stretchable components, thereby resulting in lower product and manufacturing costs. This detailed description of the present invention will include a description of a representative disposable absorbent garment including the various components of such garments. The description of the representative disposable absorbent garment will also include a description of the features encompassed by the present invention.

Representative Disposable Absorbent Garment

The disposable absorbent garments of the present invention will be described in terms of a disposable absorbent garment that is adapted to be worn by infants about the lower torso, that is, a disposable absorbent garment that is similar to a disposable diaper. It is understood that the features of the present invention are equally adaptable for other types of disposable absorbent garments such as adult incontinence garments, training pants, disposable swim pants and feminine hygiene garments.

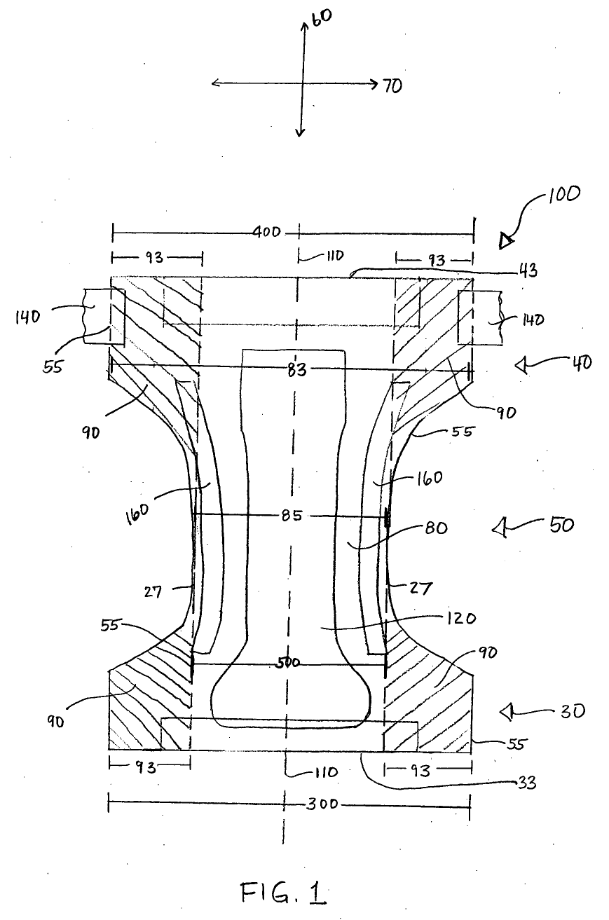

FIG. 1 representatively illustrates a disposable absorbent garment of the invention in a flat, laid-out configuration, as generally indicated at 100. The disposable absorbent garment 100 has a longitudinal direction 60 aligned with the length of the disposable absorbent garment 100 and a lateral direction 70 aligned with the width of the disposable absorbent garment 100. The lateral direction 70 is generally perpendicular to the longitudinal direction 60. The width of the disposable absorbent garment 100 may be divided in half by a lateral centerline 110 that is shown running the length of the garment. The disposable absorbent garment 100 generally defines a front waist region 30, a back waist region 40 and a crotch region 50 in between the front waist region 30 and the back waist region 40. The disposable absorbent garment 100 has two longitudinal side edges 55 and two waist edges 33 (front waist region 30 edge) and 43 (back waist region 40 edge). The waist regions 30 and 40 define widths 300 (front waist region 30 width) and 400 (back waist region 40 width) in the lateral direction 70. The crotch region 50 defines a width 500 in the lateral direction 70 that is less than the widths 300 and 400 of the waist regions 30 and 40. The disposable absorbent garments 100 may have an overall rectangular shape, T-shape or an approximately hourglass shape. In FIG. 1, the disposable absorbent garment 100 has a generally I-shape in an unfastened configuration. When the garments 100 are being worn, the longitudinal side edges 55 in the crotch region 50 may form openings for the wearer's legs and the waist edges 33 and 43 may form a waist opening around the wearer's torso.

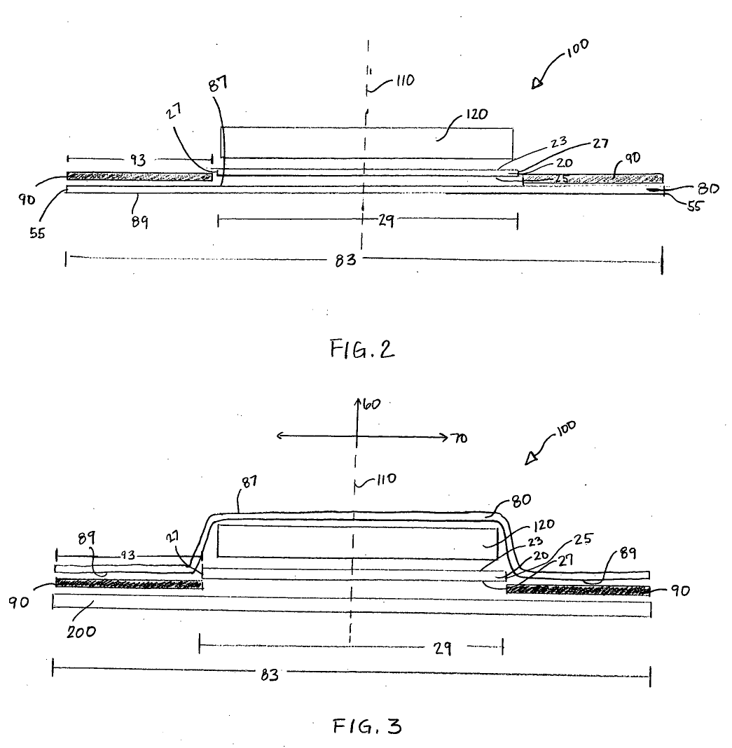

FIGS. 2-5 representatively illustrate cross-sectional views of the waist regions 300 or 400 of various aspects of disposable absorbent garments 100 of the invention. As shown in FIG. 2, the disposable absorbent garments 100 may include a liquid barrier layer 20. The liquid barrier layer 20 may define an inner surface 23, an outer surface 25 and opposite longitudinal side edges 27. The inner surface 23 is the surface of the liquid barrier layer 20 that may be oriented toward the skin or body-facing surface of the garment while the outer surface 25 may be oriented toward the clothing-facing surface of the garment. The liquid barrier layer 20 may have a liquid barrier width 29 between the opposite longitudinal side edges 27 that is approximately the same as the crotch region width 500. The liquid barrier width 29 may be slightly less that the crotch region width 500. The liquid barrier layer 20 may be manufactured from a thin plastic film or other flexible liquid-impermeable material. For example, the liquid barrier layer 20 may be formed from a polyethylene film having a thickness of from about 0.013 millimeter (0.5 mil) to about 0.051 millimeter (2.0 mils). The liquid barrier layer 20 may also be formed of a laminate of materials, such as a laminate of a film material and a nonwoven material. The materials of the liquid barrier layer 20 may be thermally or adhesively laminated together. Suitable laminate adhesives, which can be applied continuously or intermittently as beads, a spray, parallel swirls, or the like, can be obtained from Bostik-Findley, Inc., of Wauwatosa, Wis., U.S.A., or from National Starch and Chemical Company, Bridgewater, N.J., U.S.A. As an example of a thermal laminate of materials, the liquid barrier layer 20 may be formed from a stretch-thinned polypropylene film having a thickness of about 0.015 millimeter (0.6 mil) thermally laminated to a spunbond web of polypropylene fibers. The polypropylene fibers may have a fiber diameter of about 15 to microns and the nonwoven web may have a basis weight of about 17 grams per square meter (0.5 ounce per square yard). The liquid barrier layer 20 may include bicomponent fibers such as polyethylene/polypropylene bicomponent fibers.

The liquid barrier layer 20 may be composed of a micro-porous “breathable” material which permits vapors to escape while still preventing liquid exudates from passing through the liquid barrier layer 20. For example, the liquid barrier layer 20 may include a vapor permeable non-woven facing layer laminated to a micro-porous film. Suitable “breathable” liquid barrier materials are described in U.S. Pat. No. 5,695,868 issued Dec. 9, 1997 to McCormack et al. and U.S. Pat. No. 5,843,056 issued Dec. 1, 1998 to Good et al., the descriptions of which are hereby incorporated by reference. Still further, the liquid barrier layer 20 may also be an elastomeric material such as a stretch-thermal laminate (STL), neck-bonded laminate (NBL) or stretch-bonded laminate (SBL) material. Methods of making such materials are well known to those skilled in the art and are described in U.S. Pat. No. 4,663,220 issued May 5, 1987 to Wisneski et al., U.S. Pat. No. 5,226,992 issued Jul. 13, 1993 to Morman and European Patent Application No. EP 0 217 032 published on Apr. 8, 1987 in the names of Taylor et al., the disclosures of which are hereby incorporated by reference. The liquid barrier layer may also be formed of an elastic film that is also breathable as described in U.S. patent application Ser. No. 10/703,761 filed on Nov. 7, 2003 and titled “Microporous Breathable Elastic Films, Methods of Making Same, And Limited Use or Disposable Product Applications”, the disclosure of which is hereby incorporated by reference.

The disposable absorbent garments 100 of the invention may also include a stretchable carrier layer 80 placed in facing relationship with the liquid barrier layer 20. The stretchable carrier layer 80 may be directly next to the liquid barrier layer 20 or there may be other layers or materials in between the stretchable carrier layer 80 and the liquid barrier layer 20. The stretchable carrier layer 80 may have a width 83 in the waist regions 30 and 40 that is about the same as the waist region widths 300 and 400. The stretchable carrier layer 80 may also have a width 85 in the crotch region 50 that is about the same as the crotch region width 500. Desirably, the stretchable carrier layer width 83 in the waist regions defines the waist regions widths 300 and 400 of the garment 100. Put differently, the stretchable carrier layer width 83 is equal to or greater than the widths of any of the other garment components. The stretchable carrier layer 80 may be formed of a stretchable material, such as an extensible material or an elastic material. Stretchable materials are generally understood to be materials that are capable of extending from their original length when a pulling force is applied. Extensible materials generally include materials that are capable of elongating but that do not completely retract to their original length when the pulling force is removed. Extensible materials generally experience a degree of permanent deformation after elongation. Elastic materials generally include materials that are elongatable and capable of returning to substantially their original length when a pulling force is removed. The stretchable carrier layer 80 may be stretchable in the lateral direction 70 or in both the lateral direction 70 and the longitudinal direction 60. The stretchable carrier layer 80 may be formed from one or more materials including nonwoven materials, film materials and elastomers. The materials used to form the stretchable carrier layer 80 may be stretchable in a machine direction, a cross-direction or both a machine direction and a cross-direction. The machine direction of the materials may correspond to the longitudinal direction 60 of the garment 100 and the cross-direction of the materials may correspond to the lateral direction 70 of the garment 100.

The stretchable carrier layer 80 may be formed from one or more extensible materials such as those described that are described in U.S. Pat. No. 6,610,383 entitled “Transversely Extensible and Retractable Necked Laminate of Non-Elastic Sheet Layers” which issued on Aug. 26, 2003 to Morman et al. and U.S. Pat. No. 6,632,212 entitled “Breathable Laminate Permanently Conformable to the Contours of A Wearer” which issued on Oct. 14, 2003 to Morman et al. (the disclosures of which are incorporated by reference). More specifically, the stretchable carrier layer 80 may be formed of an extensible nonwoven material, such as a necked spunbond material. Alternatively, the stretchable carrier layer 80 may be formed of a laminate of an extensible film and an extensible nonwoven material (such as a necked spunbond material).

The stretchable carrier layer 80 may also be formed from one or more elastic materials including elastic nonwoven materials and laminates of nonwoven and elastic materials. The stretchable carrier layer 80 may also be formed from elastically stretchable film materials. Exemplary elastic films that are breathable are described in U.S. patent application Ser. No. 10/703,761 filed on Nov. 7, 2003 and titled “Microporous Breathable Elastic Films, Methods of Making Same, And Limited Use or Disposable Product Applications” (which has also been identified as describing suitable materials for the liquid barrier layer 20). Suitable elastic nonwoven materials include elastomeric materials that are treated using nonwoven manufacturing processes such as meltblowing. Suitable elastomers that may be formed into microfibers/nonwoven webs are described in U.S. Pat. No. 4,663,220 issued to Wisneski et al. on May 5, 1987 and titled “Polyolefin-Containing Extrudable Compositions and Methods for Their Formulation Into Elastomeric Products Including Microfibers” (incorporated by reference above). Meltblowing of KRATON copolymers (“KRATON” is a trade designation of the Shell Chemical Company) to form composite nonwoven elastic webs is described in U.S. Pat. No. 4,657,802 issued to Morman on Apr. 14, 1987 and titled “Composite Nonwoven Elastic Web”, the disclosure of which is hereby incorporated by reference. The stretchable carrier layer 80, when desirable, may also be formed from elastically stretchable laminate materials. U.S. Pat. No. 4,657,802 to Morman also describes composite nonwoven elastic webs, such as spunbond laminate (hereinafter “SBL”) webs. Other elastically stretchable laminate materials include necked bonded materials (hereinafter “NBL”) materials as are described in U.S. Pat. No. 5,226,992 issued on Jul. 13, 1993 to Morman (incorporated by reference above). Additionally, suitable breathable elastic film laminates are described in Provisional U.S. patent application Ser. No. 60/518,100 filed on Nov. 7, 2003 and titled “Microporous Breathable Elastic Film Laminates, Methods of Making Same, and Limited Use or Disposable Product Applications”, the disclosure of which is hereby incorporated by reference. Further, suitable elastic laminates are also described in a U.S. Patent Application (serial number not assigned) filed on Dec. 22, 2003 and titled “Extensible and Stretch Laminates and Method of Making Same” having attorney reference number “K-C 20,006”.

The disposable absorbent garments 100 of the invention may also include one or more elastic layers 90 attached to the stretchable carrier layer 80. The elastic layer 90 may have an elastic layer width 93 that may be defined as one elastic layer 90 edge to the other in the lateral direction 70. For example, the elastic layer 90 may begin at a longitudinal side edge 27 of the liquid barrier layer 20 and go to a longitudinal side edge 55 of the disposable absorbent garment 100. The elastic layer width 93 may extend away from the lateral centerline 110 from one longitudinal side edge 27 of the liquid barrier layer 20 toward the longitudinal side edge 55 of the garment 100 that is on the same side of the lateral centerline 110. Therefore, the elastic layer 90 is typically not in superposed relationship with the liquid barrier layer 20. The disposable absorbent garment 100 shown in FIG. 1 has four elastic layer 90 sections, two in the front waist region 30 and two in the back waist region 40. The elastic layer width 93 for each elastic layer 90 section is shown in the figure. The elastic layer 90 may be formed from known elastic materials, such as NBL or SBL materials. The various elastic materials described as being suitable for the stretchable carrier layer 80 may also suitable be used to form the elastic layer 90. The form of attachment between the elastic layer 90 and the stretchable carrier layer 80 depends on the materials selected to form the layers. The elastic layer 90 and the stretchable carrier layer 80 may inherently adhere together or they may be attached to each other by adhesive, such as a resilient adhesive that will not restrict the “stretch” properties of the layers. Elastic adhesives are known and may include elastomeric, hot melt, pressure-sensitive adhesives that are available from Bostik-Findley, Inc., Wauwatosa, Wis. under the trade designations HX-2695-01, H2503, and H2504. The combination of the elastic layer 90 and the stretchable carrier layer 80 may provide stretchable regions within the disposable absorbent garments 100 that improve the fit of the garments 100. The combination of the elastic layer 90 and the stretchable carrier layer 80 may provide stretch to the garments 100 in locations that improve the ease with which the waist regions 30 and 40 may be brought together and fastened together during wear of the garments 100. Garments 100 including the combination of the elastic layer 90 attached to the stretchable carrier layer 80 may also have a broader range of fit (meaning a wider range of body shapes and sizes may be accommodated by a given size of garment). When the stretchable carrier layer 80 is formed of a stretchable material, the elasticity of the elastic layer 90 is not inhibited or restricted.

The disposable absorbent garments 100 of the invention may also include an absorbent section 120 that may be disposed on the inner surface 23 of the liquid barrier layer 20. The absorbent section 120 may be located directly next to the inner surface 23 of the liquid barrier layer 20 or there may be other materials in between the absorbent section 120 and the liquid barrier layer 20. However, the absorbent section 120 is nearest the inner surface 23 of the liquid barrier layer 20 (as opposed to the outer surface 25) in order for the liquid barrier layer 20 to perform its function of containing liquids exuded by the wearer of the garment 100 within the absorbent section 120. The absorbent section 120 may suitably include a matrix of hydrophilic fibers, such as a web of cellulosic fluff, mixed with particles of a high-absorbency material commonly known as superabsorbent material. For example, the absorbent section 120 may include a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent section 120 may include a laminate of fibrous webs and superabsorbent material or other suitable matrix for maintaining a superabsorbent material in a localized area. The size and the absorbent capacity of the absorbent section 120 should be compatible with the size of the intended wearer and the liquid loading imparted by the intended use of the disposable absorbent garment 100. Further, the size and the absorbent capacity of the absorbent section 120 may be varied to accommodate wearers ranging from infants through adults.

The high-absorbency material may be selected from natural, synthetic, and modified natural polymers and materials. The high-absorbency materials may be inorganic materials, such as silica gels, or organic compounds, such as crosslinked polymers. The term “crosslinked” refers to methods for effectively rendering normally water-soluble materials substantially water insoluble but swellable. Such methods include, for example, physical entanglement, crystalline domains, covalent bonds, ionic complexes and associations, hydrophilic associations such as hydrogen bonding, and hydrophobic associations or Van der Waals forces. Examples of synthetic, polymeric, high-absorbency materials include the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrrolidone), poly(vinyl morpholinone), poly(vinyl alcohol), and mixtures and copolymers thereof. Further polymers suitable for use in the absorbent section 120 include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthan gum, locust bean gum and similar gums. Mixtures of natural and wholly or partially synthetic absorbent polymers may also be useful. The high absorbency material may be in any of a wide variety of geometric forms. As a general rule, the high absorbency material is in the form of discrete particles. However, the high absorbency material may also be in the form of fibers, flakes, rods, spheres or needles. In general, the high absorbency material is present in the absorbent section 120 in an amount of from about 5 to about 90 percent by weight, desirably in an amount of at least about 30 percent by weight, and even more desirably in an amount of at least about 50 percent by weight based on a total weight of the absorbent section 120. For example, in a particular aspect, the absorbent section 120 may include a laminate which includes at least about 50 percent by weight and desirably at least about 70 percent by weight of high-absorbency material overwrapped by a fibrous web or other suitable material for maintaining the high-absorbency material in a localized area. An example of high-absorbency material suitable for use in the present invention is DRYTECH 2035 polymer available from Dow Chemical, a business having offices in Midland, Mich. Other suitable superabsorbents may include FAVOR SXM 880 polymer obtained from Stockhausen, a business having offices in Greensboro, N.C.

The absorbent section 120 may include additional layers or materials such as a surge material or a spacer layer. A surge material may be located near the bodyfacing surface of the absorbent section 120 so as to receive exudates of the wearer and to provide controlled dispersion into the portion of the absorbent section 120 containing the absorbent storage materials. A spacer layer may be located near the “outer” (clothing-facing) surface of the absorbent section 120 to reduce the perception of dampness on the outside of the garment 100.

While various aspects of the present invention will be described herein, the disposable absorbent garments 100 of the invention may include additional components such as mechanical fasteners 140, leg elastics 160 and waist elastics (not shown in FIG. 1). The mechanical fasteners 140 may releasably engage the longitudinal side edges 55 of the garment 100 in the opposite waist regions 30 and 40. The mechanical fasteners 140 may include a variety of materials and surfaces known for mechanical engagement such as buttons, pins, snaps, adhesive tape fasteners, cohesives, mushroom-and-loop fasteners and hook and loop fasteners. Further, the disposable absorbent garment 100 may include an attachment panel (not shown in the figures) located on the front or back waist region 30 and 40, opposite the fasteners 140 to which the fasteners 140 may be releasably engaged during use of the garment 100. The various components of the disposable absorbent garment 100 may be integrally assembled together employing various bonding techniques, such as adhesives, ultrasonic and thermal or combinations of these techniques. If adhesive bonding is used, the adhesive may be applied as a uniform continuous layer of adhesive, a patterned layer of adhesive, a sprayed pattern of adhesive, or an array of separate lines, swirls or dots of adhesive. Desirably, the components are assembled together using ultrasonic bonding for reduced manufacturing costs.

The present invention includes various configurations of the stretchable carrier layer 80 and the elastic layer 90 in relation to the liquid barrier layer 20 and the absorbent section 120. FIG. 2 representatively illustrates a cross-sectional view taken through a waist region 30 or 40 of a garment 100 of the invention. In FIG. 2, the stretchable carrier layer 80 is in facing relationship with the outer surface 25 of the liquid barrier layer 20. The stretchable carrier layer 80 defines an inner surface 87 and an outer surface 89. The inner surface 87 of the stretchable carrier layer 80 faces the interior of the garment 100 and is therefore toward the “bodyfacing” or skin-facing surface of the garment 100. The outer surface 89 of the stretchable carrier layer 80 faces the exterior of the garment 100 and is therefore toward the clothing-facing surface of the garment 100. With the configuration illustrated in FIG. 2, the absorbent section 120 is disposed on the inner surface 87 of the stretchable carrier layer 80. With similar configurations, the absorbent section 120 may be immediately next to the inner surface 87 of the stretchable carrier layer 80 or there may be other components in between the absorbent section 120 and the inner surface 87. In FIG. 2, the liquid barrier layer 20 is in between the absorbent section 120 and the inner surface 87 of the stretchable carrier layer 80. The configuration shown in FIG. 2 includes an elastic layer 90 on both sides of the lateral centerline 110. The elastic layers 90 are immediately next to the inner surface 87 of the stretchable carrier layer 80. As shown in FIG. 2, the stretchable carrier layer width 83 defines the full extent of the width of the garment in the waist regions 30 or 40. With this configuration, the stretchable carrier layer 80 is the outermost layer of the garment 100 and its location generally corresponds with the “outer covers” of conventional disposable absorbent garments, such as disposable diapers.

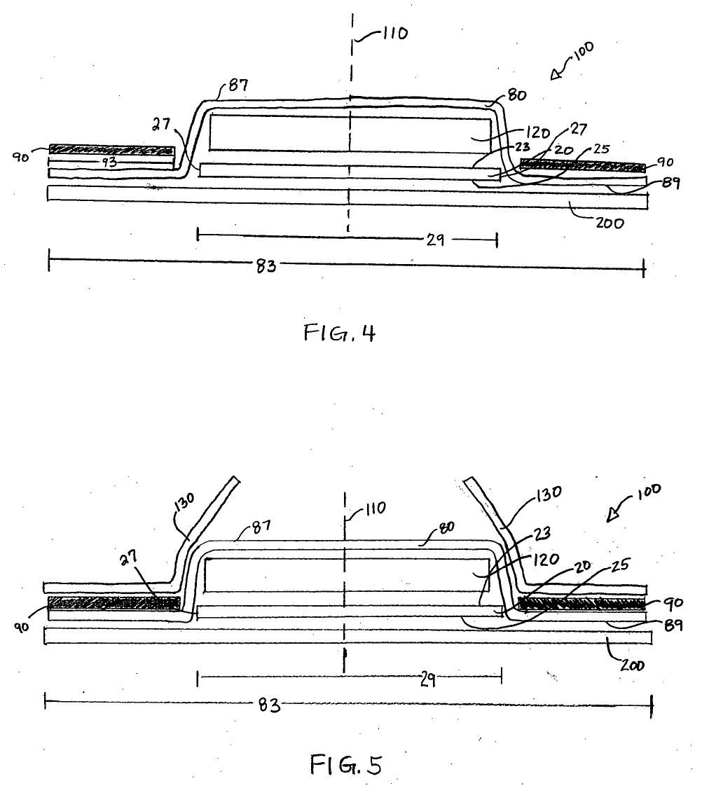

A different aspect or configuration for garments 100 of the invention is representatively illustrated in FIGS. 3 and 4. With these configurations, the stretchable carrier layer 80 defines an inner surface 87 and an outer surface 89 and the absorbent section 120 is disposed between the inner surface 23 of the liquid barrier layer 20 and the outer surface 89 of the stretchable carrier layer 80. The configurations of FIG. 3 and FIG. 4 both may include an outer cover 200. The outer cover 200 may be included in order to provide a unitary appearance to the garment 100. The outer cover 200 may be formed of a nonwoven material (such as a spunbond material) or of a laminate material (such as a nonwoven-film laminate). The outer cover 200 may provide liquid barrier properties to the garment 100. In both FIG. 3 and FIG. 4, the stretchable carrier layer 80 is the component that provides the body or skin-facing surface for the garment 100. The stretchable carrier layer 80 may be provided with a lotion or treatment that is formulated to be transferred to the wearer's skin. Suitable compositions for application to the stretchable carrier layer 80 are described in U.S. Pat. No. 6,149,934 that issued to Krzysik et al. on Nov. 21, 2000, the disclosure of which is hereby incorporated by reference. The stretchable carrier layer 80 may also be treated with a surfactant in order to increase its wettability and to improve the intake of fluids into the absorbent section 120. The material used to form the stretchable carrier layer 80 may be surface treated with about 0.3 weight percent of a surfactant, such as a surfactant commercially available from Hodgson Textile Chemicals, Inc. under the trade designation AHCOVEL Base N-62. With the configurations shown in FIGS. 3 and 4, the stretchable carrier layer 80 has a stretchable carrier layer width 83 in the lateral direction 70. The stretchable carrier layer width 83 in the lateral direction 70 is equal to or greater than the width of the outer cover 200.

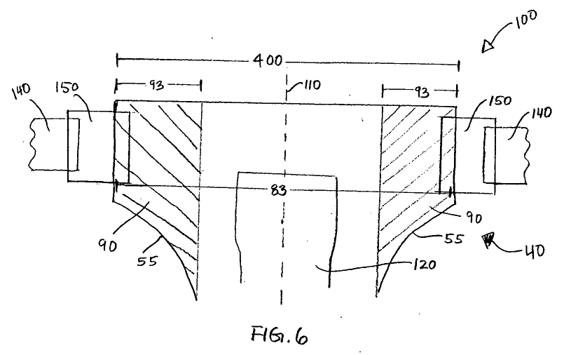

In FIG. 3, the elastic layer 90 is attached to the outer surface 89 of the stretchable carrier layer 80. With this configuration, the elastic layer 90 is between the stretchable carrier layer 80 and the outer cover 200. In FIG. 4, the elastic layer 90 is attached to the inner surface 87 of the stretchable carrier layer 80. Both configurations may provide a garment 100 having stretchable regions that improve the fit of the garments around the waist of a wearer when the waist regions 30 and 40 are joined by a fastener 140. Desirably, with the configuration shown in FIG. 3, the outer cover 200 is formed of a stretchable material so that the elasticity of the elastic layers 90 is not inhibited or restricted. An additional aspect of the present invention is representatively illustrated in FIG. 5. The disposable absorbent garments 100 of the invention may further include a stretchable containment flap layer 130. The stretchable containment flap layer 130 may be attached to the inner surface 87 of the stretchable carrier layer 80. With the configuration of FIG. 5, the elastic layers 90 are between the stretchable carrier layer 80 and the stretchable containment flap layers 130. Depending on the material selected to form the elastic layer 90, the elastic layer 90 may provide the attachment between the stretchable containment flap layer 130 and the stretchable carrier layer 80. The stretchable containment flap layer 130 may be formed from one or more of the stretchable materials already identified herein. When the stretchable containment flap layer 130 is formed of a stretchable material, the elasticity of the elastic layer 90 is not inhibited or restricted.

As described herein, the combination of the elastic layer 90 and the stretchable carrier layer 80 provide garments 100 having improved fit and improved range of fit. In order to further improve the range of fit provided by a given garment size, the disposable absorbent garments 100 of the invention may include an elastic ear material 150. The elastic ear material 150 may be bonded to a longitudinal side edge of the elastic layer 90. This aspect of the present invention is representatively illustrated in FIG. 6. The elastic ear material 150 may have a fastener element 140 attached to it. The elastic ear material 150 provides a greater range of extension for bringing the back waist region 40 of the garment 100 into contact with the front waist region 30. In addition to being bonded to the elastic layer 90, the elastic ear material 150 may also be simultaneously bonded to the stretchable carrier layer 80 and the outer cover 200. The fastener element 140 may be adhesively, ultrasonically or thermally bonded to the elastic ear material 150. The elastic ear material 150 may be selected from one or more of the elastic materials identified herein.

The present invention also relates to methods of making the disposable absorbent garments 100 already described herein. The methods of the invention differ from existing methods for making garments at least with respect to how the elastic layer 90 is integrated with the other components of the garments 100. For example, the elastic layer 90 is attached to a stretchable carrier layer 80 that runs the full width of the garment 100 in the lateral direction 70. The present invention includes a method of making disposable absorbent garments 100 that have a front waist region 30, a back waist region 40 and a crotch region 50 that extends between and connects the waist regions 30 and 40. The disposable absorbent garments 100 include a longitudinal direction 60 and a lateral direction 70 that is generally perpendicular to the longitudinal direction 60. The method includes a step of providing a first continuous web of material. The first continuous web may include a stretchable carrier layer 80 and intermittent elastic material segments. The intermittent elastic material segments form the elastic layer(s) 90 in the finished garments 100. The stretchable carrier layer 80 may be formed of one or more of the stretchable materials already described herein. The intermittent elastic material segments may be provided in generally rectangular-shaped patches near one or both of the longitudinal side edges of the first continuous web. The intermittent elastic material segments may be provided in such a way that the longer side of the rectangular shape is parallel to the longitudinal side edge of the first continuous web. The dimensions of the intermittent elastic material segments and their frequency in the first continuous web may be selected considering the desired placement of elastic layers 90 and the desired elastic layer width 93 in the finished garments 100. Examples of possible placements are: (1) elastic layer 90 in the back waist region 40 of the finished garment 100, where the garments 100 are manufactured back waist region 40 to front waist region 30; (2) elastic layer 90 in the back waist region 40 of the finished garment 100, where the garments 100 are manufactured back waist region 40 to back waist region 40; (3) elastic layer 90 in the back waist region 40 and the front waist region 30 of the finished garment 100, where the garments 100 are manufactured back waist region 40 to front waist region 30; and (4) elastic layer 90 in the back waist region 40 and the front waist region 30 of the finished garment 100, where the garments 100 are manufactured back waist region 40 to back waist region 40. The dimensions of the intermittent elastic material segments are such that the intermittent elastic material segments would not generally cover the entire width of the first continuous web. The intermittent elastic material segments may be applied to the stretchable carrier layer 80 using a function roll. Suitable function rolls are known in the art and are described in International Publication No. WO 03/037769 titled “Apparatus and Method For Applying Discrete Components Onto A Moving Web” and U.S. Pat. No. 5,716,478 issued to Boothe et al. on Feb. 10, 1998 and titled “Apparatus and Method For Applying Discrete Parts Onto A Moving Web”. Alternatively, a timed, slip-cutting vacuum roll may be used to apply the intermittent elastic material segments to the stretchable carrier layer 80.

The method may also include a step of providing a second continuous web. The second continuous web may include a liquid barrier material such as those described herein as being suitable for the liquid barrier layer 20. The method may further include a step of joining the first continuous web in facing relationship with the second continuous web to form an outer cover web. The first continuous web and the second continuous web may be joined to each other by adhesives, ultrasonic bonding or thermal bonding. The second continuous web may be narrower than the first continuous web. After the first continuous web and the second continuous web are joined to form an outer cover web, the method may include a step of providing a series of absorbent assemblies and attaching the absorbent assemblies to the outer cover web to form a garment web. The next step in the method may then be selective cutting of the garment web into individual garments. The garment web may be selectively cut in such a way that the intermittent elastic material segments are generally bisected across the width of the garment web where the width of the garment web corresponds to the lateral direction of the finished garments 100. The absorbent assemblies may include an absorbent section 120 for absorbing and containing bodily exudates of the wearers of the garments 100. The absorbent assemblies may be attached to the outer cover web by adhesives, ultrasonic bonding or thermal bonding. After the absorbent assemblies are attached to the outer cover web, the resulting garment web may have components in the following orders (in order from what forms the body or skin-facing surface of the final garments 100 toward the clothing-facing surface of the garments 100): (1) absorbent assembly, first continuous web and second continuous web; and (2) absorbent assembly, second continuous web and first continuous web. The disposable absorbent garments 100 of the invention may be made in back waist region to back waist region formation or in back waist region to front waist region formation. When the intermittent elastic material segments are bisected and the formation is either “back to back” or “back to front”, the individual garments 100 will have at least one elastic layer 90 in the back waist region 40 and may have at least one elastic layer 90 in the front waist region 30. With some aspects, the method may include an additional step of providing a liner web in facing relationship with the absorbent assemblies prior to the selective cutting of the garment web. The liner web may form the body or skin-facing surface in the finished garments 100.

In another aspect of the method of the present invention, the method provides for formation of disposable absorbent garments 100 in which the absorbent assembly is located in between a first continuous web including a liquid barrier material and a second continuous web. The method includes providing a first continuous web where the first continuous web includes a liquid barrier material and the first continuous web has an outer surface and an inner surface. The outer surface of the first continuous web may correspond to the clothing-facing surface of the finished garment 100 and the inner surface may correspond to the body or skin-facing surface of the finished garment 100. The method may also include a step of providing a series of absorbent assemblies and attaching the absorbent assemblies to the inner surface of the first continuous web. The method may next include a step of providing a second continuous web in facing relationship with the inner surface of the first continuous web to form an article web. The second continuous web may include a stretchable carrier layer and intermittent elastic segments. Therefore, the absorbent assembly is provided in between the first continuous web and the stretchable carrier layer 80. The stretchable carrier layer 80 may define the width in the lateral direction of the finished garments 100. The stretchable carrier layer 80 may have an outer surface 89 and an inner surface 87 (similar to the orientation of the surfaces of the first continuous web) and the intermittent elastic segments may be attached to either the outer surface 89 or the inner surface 87. The method may include an additional step of providing a stretchable containment flap web in facing relationship with the inner surface 87 of the stretchable carrier layer 80 prior to the garment web being selectively cut into individual disposable absorbent garments 100.

The disposable absorbent garments and methods of forming them, as encompassed by the present invention, provide good fit properties while eliminating the need to attach separate stretchable ear portions. As a result, the disposable absorbent garments 100 of the invention may have lower raw material costs and may be produced at higher rates. While the disposable absorbent garments 100 of the invention have been described in detail with respect to specific aspects thereof, it will be appreciated that those skilled in the art, upon attaining an understanding of the foregoing, may readily conceive of alterations to, variations of and equivalents to these garments. Accordingly, the scope of the present invention should be assessed as that of the appended claims and any equivalents thereto.

1. A disposable absorbent garment having a longitudinal direction and a lateral direction; a front waist region, a back waist region and a crotch region that connects the front waist region and the back waist region; a waist region width in the lateral direction and a crotch region width in the lateral direction that is less than the waist region width, and two longitudinal side edges, the disposable absorbent garment comprising:
a liquid barrier layer defining an inner surface, an outer surface, opposite longitudinal side edges and a liquid barrier layer width between the opposite longitudinal side edges of about the crotch region width;
a stretchable carrier layer placed in facing relationship with the liquid barrier layer, the stretchable carrier layer having a width in the waist regions of about the waist region width and a width in the crotch region of about the crotch region width;
an elastic layer attached to the stretchable carrier layer, the elastic layer having an elastic layer width extending away from a lateral centerline of the disposable absorbent garment in the lateral direction from a liquid barrier layer longitudinal side edge to a disposable absorbent garment longitudinal side edge; and
an absorbent section disposed -on the inner surface of the liquid barrier layer.

2. The disposable absorbent garment of claim 1, wherein the stretchable carrier layer defines an inner surface and an outer surface and the absorbent section is disposed on the inner surface of the stretchable carrier layer.

3. The disposable absorbent garment of claim 1, wherein the stretchable carrier layer defines an inner surface and an outer surface and the absorbent section is disposed between the inner surface of the liquid barrier layer and the outer surface of the stretchable carrier layer.

4. The disposable absorbent garment of claim 3, wherein the elastic layer is attached to the inner surface of the stretchable carrier layer.

5. The disposable absorbent garment of claim 3, wherein the elastic layer is attached to the outer surface of the stretchable carrier layer.

6. The disposable absorbent garment of claim 4, further comprising a stretchable containment flap layer attached to the inner surface of the stretchable carrier layer.

7. The disposable absorbent garment of claim 1, wherein an elastic layer is attached to the stretchable carrier layer on both sides of the lateral centerline in the back waist region.

8. The disposable absorbent garment of claim 1, wherein an elastic layer is attached to the stretchable carrier layer on both sides of the lateral centerline in the back waist region and the front waist region.

9. The disposable absorbent garment of claim 1, wherein the stretchable carrier layer is extensible in the lateral direction.

10. The disposable absorbent garment of claim 1, wherein the elastic layer has a fastener element attached.

11. The disposable absorbent garment of claim 7, wherein an elastic ear material is bonded to a longitudinal side edge of the elastic layer.

12. The disposable absorbent garment of claim 11, wherein the elastic ear material has a fastener element attached.

13. A method of making a disposable absorbent garment that defines a front waist region, a back waist region, a crotch region that extends between and connects the waist regions, a longitudinal direction and a lateral direction, the method comprising:
a) providing a first continuous web, the first continuous web comprising a stretchable carrier layer and intermittent elastic material segments;
b) providing a second continuous web, the second continuous web comprising a liquid barrier material;
c) joining the first continuous web in facing relationship with the second continuous web to form an outer cover web;
d) providing a series of absorbent assemblies and attaching the absorbent assemblies to the outer cover web to form a garment web; and
e) selectively cutting the garment web into individual garments, generally bisecting the intermittent elastic material segments in the lateral direction.

14. The method of claim 13, further comprising the additional step of providing a liner web in facing relationship with the absorbent assemblies prior to selectively cutting the garment web.

15. A method of making a disposable absorbent garment that defines a front waist region, a back waist region, a crotch region that extends between and connects the waist regions, a longitudinal direction and a lateral direction, the method comprising:
a) providing a first continuous web, the first continuous web comprising a liquid barrier material and having an outer surface and an inner surface;
b) providing a series of absorbent assemblies and attaching the absorbent assemblies to the inner surface of the first continuous web;
c) providing a second continuous web in facing relationship with the inner surface of the first continuous web to form a garment web, the second continuous web comprising a stretchable carrier layer and intermittent elastic material segments; and
e) selectively cutting the garment web into individual garments, generally bisecting the intermittent elastic material segments in the lateral direction.

16. The method of claim 15, wherein the stretchable carrier layer has an outer surface and an inner surface and the intermittent elastic material segments are attached to the inner surface.

17. The method of claim 15, wherein the stretchable carrier layer has an outer surface and an inner surface and the intermittent elastic material segments are attached to the outer surface.

18. The method of claim 16, further comprising the additional step of providing a stretchable containment flap web in facing relationship with the inner surface of the stretchable carrier layer prior to selectively cutting the garment web.
